(19) United States
(12) Patent Application Publication
(10) Pub. No.: US 2022/0338801 A1
(43) Pub. Date: Oct. 27, 2022

(54) Wearable System for Automated, Objective and Continuous Quantification of Pain

(71) Applicant: THE REGENTS OF THE UNIVERSITY OF COLORADO A BODY CORPORATE, Denver, CO (US)

(72) Inventors: Tam VU, Boulder, CO (US); Robin DETERDING, Boulder, CO (US); Hoang TRUONG, Boulder, CO (US)

(21) Appl. No.: 17/642,317

(22) PCT Filed: Sep. 11, 2020
(86) PCT No.: PCT/US2020/050532

§ 371 Date: Mar. 11, 2022

Related U.S. Application Data
(60) Provisional application No. 62/900,176, filed on Sep. 13, 2019.

(51) Int. Cl. A61B 5/00; A61B 5/313; A61B 5/024; A61B 5/0533

(52) U.S. Cl. CPC A61B 5/4824; A61B 5/313; A61B 5/6803; A61B 5/02416; A61B 5/0533; G16H 20/10

(57) ABSTRACT

A wearable objective pain measuring device includes a headband configured to be worn by a user on the user's head. The device includes one or more sensors in the headband, including one or more of: a sweep impedance profiling sensor to collect data reflective of activity of corrugator supercilia during a pain session; an electromyography sensor to collect data signals from a brain that reflects pain perception; a photoplethysmogram sensor configured to collect data regarding changes in hear rate and heart rate variability due to pain state; or a galvanic skin response sensor configured to collect data related to changes in sweat gland and skin conductance due to a pain state. The device includes a pain quantification pipeline to objectively quantify pain based on data from the one or more sensors. The device includes an output device configured to output an objective pain quantification based on quantifying pain.

### CROSS-REFERENCE TO RELATED APPLICATIONS

This application claims the benefit of and priority to U.S. Provisional Patent Application Ser. No. 62/900,176 filed on Sep. 13, 2019 and entitled “WEARABLE SYSTEM FOR AUTOMATED, OBJECTIVE AND CONTINUOUS QUANTIFICATION OF PAIN,” which application is expressly incorporated herein by reference in its entirety.

### BACKGROUND

In recent decades, the United States has been facing the outbreak of chronic pain and opioid abuse, in which around 20% of the population have had persistent pain and over 40,000 lives have been lost every year due to opioid misuse. Unfortunately, about 25% of patients who have been prescribed opioids for pain misuse them, about 5% of the misusers translate to heroin, and about 10% of them develop an opioid use disorder. In addition, about 80% of people who use heroin have previously misused prescription opioids, both intentionally and unintentionally. Similar to other neuropsychological constructs, a critical challenge in pain management emerges from the fact that pain cannot be directly measured without invasively accessing the nervous system. Therefore, the current non-invasive gold standard for the assessment of pain is self-reporting where people are asked to report their pain intensity. Though self-report is a common measure that reflects a patient's conscious perception of the given painful sensation, it has many limitations, especially for long-term usage: (1) it is often subjective and unnatural. The self-reporting results is affected by the subject's cognitive load and emotion at the reporting moment, (2) it introduces bias since frequent self-reporting of pain can create pain perception, and (3) it cannot be assessed frequently or continuously as this would present an unrealistic burden on the patient.

Thus, there is a need for a more objective measure of pain that is efficacious and fits the daily real-world usage. The most accurate and objective pain quantification approach is using an fMRI machine. However, it is expensive and inconvenient since it is only available in a clinical setting. Other common biosignals used for pain-related quantification include electroencephalography (EEG), electromyography (EMG), galvanic skin response (GSR), electrocardiography (ECG) and photoplethysmogram (PPG). However, existing systems often share a limitation that prevents them from seeing their prime time for use in daily and real-world settings. The required sensors are bulky (e.g., an EEG cap) or the number of sensors required is large since the system does not directly capture the pain-related features. This leads to the difficulties in integrating them into a wearable device for daily usage.

The subject matter claimed herein is not limited to embodiments that solve any disadvantages or that operate only in environments such as those described above. Rather, this background is only provided to illustrate one exemplary technology area where some embodiments described herein may be practiced.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates an overview of a pain measuring device and its applications;

FIG. 2 illustrates how pain affects various physiological processes;

FIG. 3 illustrates a pain measuring device system flow diagram;

FIG. 4 illustrates a sensor configuration over corrugator supercilia;

FIG. 5 illustrates a pain measuring device hardware schematic;

FIG. 6 illustrates a fabricated printed circuit board including delineations of analog and digital ground planes;

FIGS. 7A-7F illustrate a pain measuring device sensor measurements;

FIG. 8 illustrates a graphical representation of impact of feature selection approaches;

FIG. 9 illustrates a graphical representation of the impact of different epoch sizes;

FIG. 10 illustrates a graphical representation of the impact of training and testing ratios;

FIG. 11 illustrates a confusion matrix showing predicted pain levels correlated with true pain levels;

FIG. 12 illustrates a graphical representation showing the impact of sensor combinations; and

FIG. 13 illustrates a method for objectively quantifying pain.

### DETAILED DESCRIPTION

### 1. Introduction

Various features of various embodiments of the invention are illustrated by reference to a particular embodiment referred to herein as Painometry. Painometry is a multimodality sensing headband for objective pain quantification. Embodiments are configured to capture core signals that are directly correlated to pain perception, such as the facial muscle activity above and between one's eyes, in combination with a small number of less pain-specific signals. The headband can be used in one or more of a wide range of applications from in-hospital scenarios such as automatically and objectively answering the pain rating assessment; assisting anesthetic monitoring; to in-home pain assessment for accurate dosing of pain relief medication; and pain management such as daily and real-time recommendation on time to take medications. Some challenges in developing such a system include: (1) capturing accurately and specifically the muscle activity affected by the autonomous nervous system during pain is difficult, (2) pain-related features from multiple biosignals that are useful in providing an objective pain quantification are unknown, (3) different types of noise present in the multitude of the signals (i.e., noise of individual sensors and cross-interference between ‘active’ and ‘passive’ sensing components).

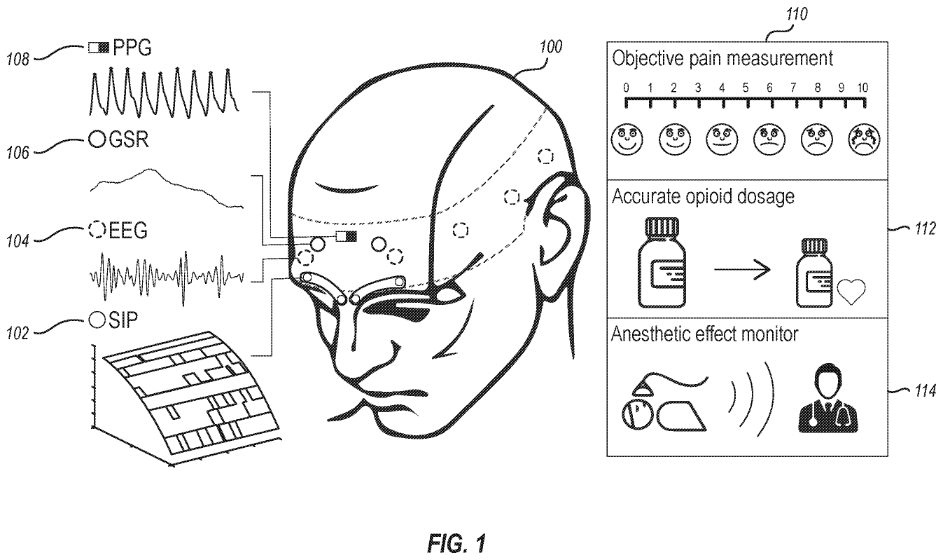

Reference is now made to FIG. 1. Aiming to capture the autonomous muscle activity in high spatial resolution with a small number of sensors, embodiments may include Sweep Impedance Profiling (SIP), a sensing technique that captures the miniature movement of under-skin muscle at the exact point where the sensor makes contact with the skin. Thus, FIG. 1 illustrates SIP sensors 102 on a patient 100. To improve the sensitivity of the system, embodiments can leverage a small number of head-based biosignals by including additional sensors including one or more electroencephalography (EEG) sensors 104 to collect data signals from a brain that reflects pain perception, one or more galvanic skin response (GSR) sensors 106 configured to collect data related to changes in sweat gland and skin conductance due to a pain state, and/or one or more photoplethysmogram (PPG) sensors 108 configured to collect data regarding changes in hear rate and heart rate variability due to pain state. FIG. 1 illustrates various locations the various sensors can be placed on the patient. To objectively quantify different pain levels (using a pain quantification module, using one or more processors and one or more computer executable instructions executed by the processors), embodiments may make use of a thorough experiment protocol and analyze recorded biosignal data to build a quantification model that utilizes pain-related feature extraction and selection. An output device (such as a computer display and user interface) can output an objective pain quantification based on training data and sensor input. Embodiments may be implemented using hardware designs which follow established safety guidelines to ensure the system will not cause any safety concerns.

### 2. Anatomy and Physiology of Pain

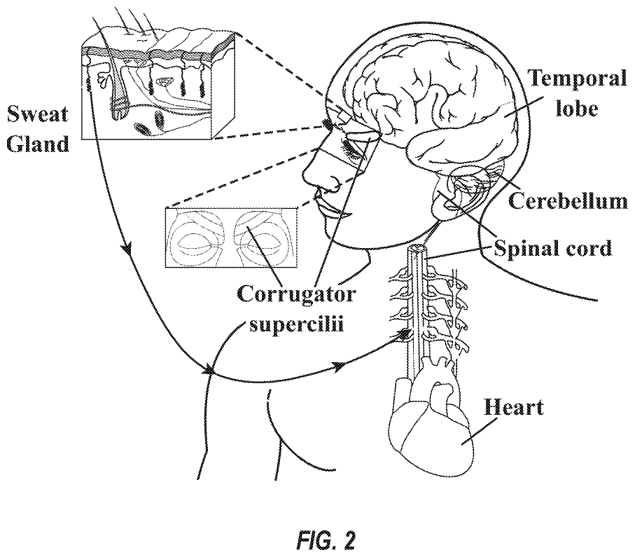

In this section the anatomy and physiology of pain is discussed to understand the activation of the autonomous nervous system (ANS) (see FIG. 2) during pain, which causes the autonomic responses of corrugator muscle tone, heart rate, skin conductance (e.g., due to sweat), and brain activity.

Pain perception. Pain is a subjective experience that results from the transduction of noxious stimuli into neural signals that are transmitted to higher cortical brain regions, resulting in a conscious, subjective interpretation of pain. The sensation of pain is the result of tightly coupled neurophysiological processes the peripheral detection of noxious stimuli and the transmission and processing of these signals in the central nervous system.

Acute pain and chronic pain. Acute pain is a short-term effect of pain (typically lasts less than 3 to 6 months) or is directly related to soft tissue damage with sharp and dull sensations. On the other hand, chronic pain lasts longer where the pain can become progressively worse and reoccur intermittently. Strong correlations exist between the severity of acute postoperative pain and the development of chronic pain. Treating acute pain is extremely important because if acute pain is not adequately treated, it may lead to the development of harder-to-treat chronic pain conditions.

Importance of objective pain quantification. To determine a patient's current pain state, physicians rely primarily on patient's subjective self-report, typically using a pain scale 110 (e.g., Visual analog scale (VAS) from 0=no pain to 10=worst pain imaginable) to assess the patient's current level of pain. A pain scale requires patients to evaluate and communicate their pain levels, and the pain score is influenced not only by the current objective level of pain but also by patients' pain tolerance, current environment, memories of past painful episodes, and willingness to communicate painful experiences.

In addition, patients have difficulty providing a good estimate of their current pain state because they have no objective way to distinguish the score. The lack of an objective pain measurement method leads to either (1) overdosing of patients and drug (e.g., opioid) misuse or (2) underdosing of patients and unnecessary pain for the patients. Thus, an objective pain quantification system brings benefits in (1) removing the need for a patient to verbalize their pain state, (2) improving dosing 112 which can reduce the risk of developing an opioid addiction or reduce patients' suffering, and (3) improving clinical and healthcare systems with reliable assessment monitoring 114 of patients' condition.

Physiological expressions which could be noninvasively sensed. The hypothalamus and limbic brain regions involved in the perception of pain are also involved in the modulation of the ANS, which is responsible for bodily functions that are regulated without conscious input, such as breathing, heart rate, vasomotor activities, and reflex reactions. The hypothalamus integrates regulatory input from the limbic system and regulates functions of the ANS. Autonomic responses to pain include brain activity, heart rate, skin conductance and facial muscle groups (i.e., corrugator supercilii, zygomaticus) depicted in FIG. 2. These autonomic responses can be captured with EEG sensors 104, PPG sensors 108, GSR sensors 106, and muscle activity sensors (such as SIP sensors 102) respectively.

With the goal of having a wearable system suitable for daily usage, one of the areas where it is feasible to accommodate all those sensors is the Fpz-Oz horizontal line in a 10-20 system of electrode placement that passes the forehead area, as illustrated by the sensor placements in FIG. 1. Some embodiments leverage the choices for those sensor montages based on the intuition that the forehead (1) provides a good measurement for GSR, (2) is a common area to measure PPG signals, and (3) covers a part of the corrugator supercilii. Since the signals resulting from muscular contractions initiated by the autonomic innervation of corrugator supercilii are typically small compared to larger signals from neighboring facial muscles (e.g., eye blinking, eye movement, and jaw movement), the passive EMG measurement with electrodes over the eyebrow is prone to noise. Thus, embodiments may implement a high spatial resolution with measurements sufficiently sensitive to capture the autonomic response of corrugator supercilii.

### 3. System Overview

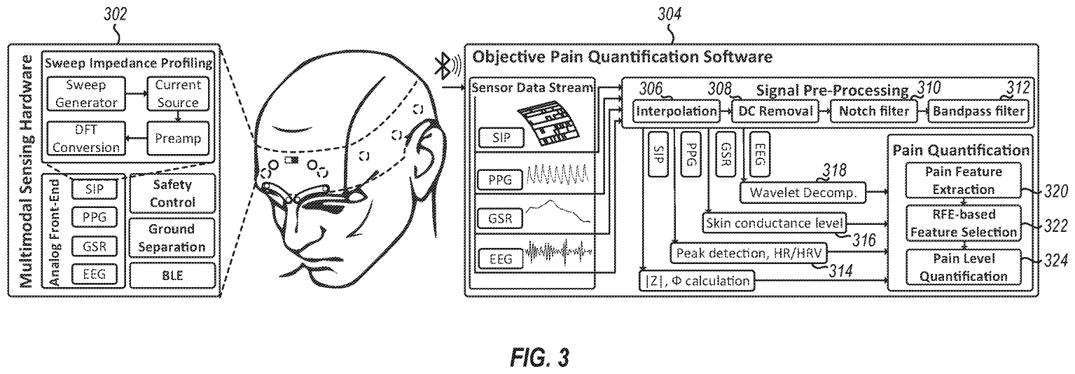

In this section, one illustrative embodiment is illustrated. The overall design of Painometry, the multimodal sensing headband for objective pain quantification based on the analysis of physiological expression of pain and autonomic responses is shown in FIG. 3. In particular, FIG. 3 illustrates sensing hardware 302 (which may include the various sensors shown in FIG. 1) along with certain circuits coupled to the sensors and Objective Pain Quantification Software 304. The software 304 may be implemented as computer executable instructions stored on one or more computer readable storage media. When the instructions are executed by one or more processors, the various functions, modules, and algorithms illustrated in the software 304 are implemented or performed. Embodiments may have certain features, including: (1) sensitive and high spatial resolution muscle activity sensing; (2) a reliable and safe multimodal physiological sensing system; and/or (3) highly accurate and light-weight pain level quantification.

Sensitive and high spatial resolution muscle activity sensing. Muscle activity is traditionally measured using the electromyography (EMG) technique. EMG sensors passively capture the biopotential at the sensor montages. However, these biopotentials are usually the combination of the biopotentials of multiple sources due to different muscle movements. One example is that the EMG sensors on the forehead also capture eye movements and blinking. In order to capture the activity of the muscle group of interest only, one of the conventional solutions is to have multiple sensors that capture biopotentials from other muscle sources and then use regression or ICA techniques to extract the necessary information. In order to capture the muscle activity of the corrugator supercilii muscle with a fewer number of sensors and to overcome the noise obstacle, embodiments may implement a sweep impedance profiling (SIP) sensing technique. By attaching only two sensors on top of the muscle group of interest, this sensing method can capture the change in impedance caused by the innervation of that muscle group.

Reliable, wearable, and safe multimodal physiological sensing system. In order to capture various physiological expressions of the human body, a conventional multimodal sensing system includes different combinations of sensing modalities such as EEG, EMG, ECG, eye tracking, facial expressions, GSR, etc. These systems are popular in mental health and cognitive related research such as stress detection, seizure prediction, and sleep monitoring. Existing pain-sensing applications analyze individual biosignals or different combinations of EEG, EMG, GSR, and PPG to explore distinctive features among different pain states. However, there are challenges in bringing all those sensors together in the same hardware form that further fits into a wearable in a daily usage scenario. In particular, integrating multiple sensors on a small-size hardware is prone to co-existing interference noise. In addition, some GSR and SIP sensors are considered ‘active’ sensors where a current runs through part of a human body. Thus, it is required to consider safety constraints. Embodiments of a multimodal sensing system including SIP, EEG, GSR, and PPG are configured to minimize the cross-interference noise and restrict the ‘active’ sensors under safety thresholds.

Highly accurate pain level quantification. One challenge in building the pain quantification system is the subjective nature of pain perception. Another challenge is determining distinctive features among different pain states. Existing work provides various findings in pain-related feature extraction from multiple sensors, however, there is no universally common feature set that can be directly employed. Some embodiments may be implemented based on results of research using an experimental pain-inducing device and data collection protocol to evaluate headband embodiments and to obtain highly accurate ground truth labels. A set of possible features is extracted from each corresponding biosignal and a recursive feature elimination is used as a feature selection algorithm, implemented by one or more processors and computer executable instructions implemented on the processors, to identify the most discriminating set of features. Finally, a classification model is trained offline and then used for online pain quantification.

### 4. Sweep Impedance Profiling

As stated in previous sections, some embodiments are designed to implement an accurate and sensitive muscle activity sensing; and minimize the number of electrode montages to fit into a wearable and daily-usage scenario. Sweep Impedance Profiling (SIP) is a technique to measure the impedance of surface muscles using multiple AC ‘active’ signals, which (1) is accurate and sensitive measurements of a single muscle group under a wide frequency range, (2) is safe, and (3) requires minimal montages. The relationship between muscle impedance and muscle length can be used for implementing designs to optimize SIP measurement.

Impedance of human body part. The impedance Z of an object is given by: Z=R+jX where R is the resistance and X is the combination of capacitance XC and inductance L of the given object. In human-body-related applications, because the inductance plays a minimal role in standard impedance measurement, X=XC=½πf C. As a result, total impedance magnitude |Z| and impedance phase ϕ of a human body part will be calculated as

$$\vert z \vert = \sqrt{{R}^{2} + {X}_{C}^{2}} , \\ ϕ = {tan}^{- 1} \frac{{X}_{C}}{R} \qquad ( 1 )$$

Relationship of muscle impedance to muscle movement. In the illustrated example, the muscle is modeled as a solid cylinder 402 shaped myofiber with length of L and radius of r. The muscle resistance and self capacitance will be calculated as follows:

$$R = ρ \frac{L}{A} , \\ C = ( 8 + 4.1 {( \frac{L}{r} )}^{0.76} ) {ε}_{m} r \qquad ( 2 )$$

where ρ is muscle resistivity coefficient, A is relative cross-cut, εₘ is overall muscle permittivity, and f is the excitation frequency.

Total impedance of the muscle at excitation frequency f will be given as:

$$Z ( f ) = \sqrt{{( ρ \frac{L}{A} )}^{2} + {( \frac{1}{2 π ( 8 + 4.1 {( \frac{L}{r} )}^{0.76} ) {ε}_{m} fr} )}^{2}} \qquad ( 3 )$$

In this equation, there are three variables, i.e., L, A and r, that will change due to muscle activity (e.g., the autonomic innervation of corrugator muscle in the illustrated scenario). Sweeping through different excitation frequencies f and obtaining corresponding Z(f) will yield a ‘profile’ of the muscle group of interest via the equation system built from Eq. 3. This equation system can be solved to find the corresponding L, A and r values at a certain muscle condition.

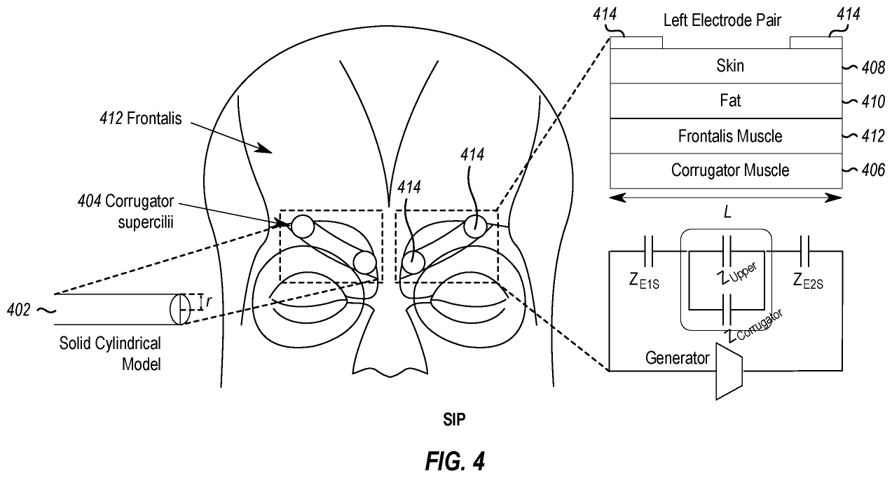

FIG. 4 shows the anatomy of facial muscle related to the corrugator supercilii area 404 under four layers of skin 408, subcutaneous fat 410, frontalis muscle 412, and corrugator muscle 406. With regard to the corrugator muscle, the corresponding basic sweeping circuit is comprised of four impedances as shown in FIG. 4. In this model, Z_{E1S} and Z_{E2S} are depended on the contact between electrodes 414 and skin and are calibrated using lead-off-detection (discussed below). Thus, sweeping f over a short period altogether with a concrete way to calculate total impedance will provide a corresponding impedance profile of this selected muscle group.

DFT-based impedance measurement. The Discrete Fourier Transform (DFT) technique correlates the signal of interest with sinusoidal basis functions, which are both sine and cosine waveforms. The correlation of the analyzed signal with the sinusoidal basis functions results in a complex numeric value, in which the imaginary and real parts represent the correlation of the signal with the sine and cosine waveforms, respectively. From this correlation value, its magnitude is used to compute the magnitude spectrum, and its phase value is used to obtain the phase spectrum. Specifically, given the voltage at generator:

vₛ(t)=V₀ cos(2πft+θ) (4)

The real RE and imaginary IM parts of each sample iϵ[ON−1] after the DFT correlation process is:

$$\{ \begin{matrix}{RE}_{i} = \frac{2}{N} {\sum }_{n = 0}^{N - 1} [ {v}_{i} ( nT ) cos ( \frac{2 π}{N} n ) ] \\ {IM}_{i} = \frac{2}{N} {\sum }_{n = 0}^{N - 1} [ {v}_{i} ( nT ) sin ( \frac{2 π}{N} n ) ]\end{matrix} \qquad ( 5 )$$

where T is sampling interval and N is the number of sample respectively.

Magnitude and phase of the unknown impedance will be calculated as:

$$\{ \begin{matrix}\vert Z \vert = \frac{\sqrt{{RE}_{Z}^{2} + {IM}_{Z}^{2}}}{\sqrt{{RE}_{R}^{2} + {IM}_{R}^{2}}} \\ ϕ = {tan}^{- 1} ( \frac{{RE}_{Z} {IM}_{R} - {RE}_{R} {IM}_{Z}}{{RE}_{Z} {RE}_{R} + {IM}_{Z} {IM}_{R}} )\end{matrix} \qquad ( 6 )$$

DFT-based measurement provides accurate impedance measurement but has large calculation and memory requirement to store the resulted RE and IM parts. Thus, compared to conventional EMG, the SIP method has a higher spatial and spectral resolution but lower temporal resolution.

### 5. Multimodal Sensing Hardware

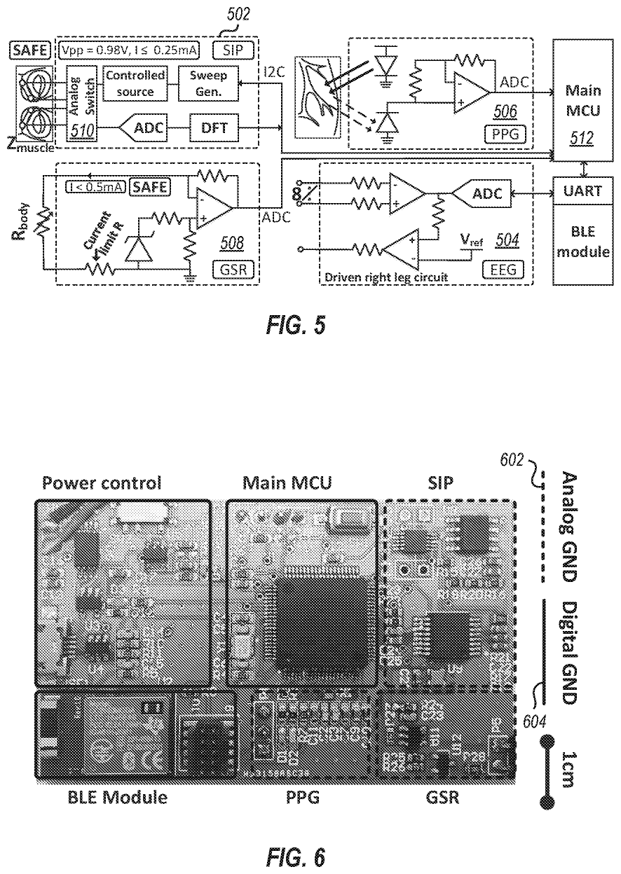

In this section, design-related issues (e.g., safety concerns and noise constraints) and corresponding solutions to integrate SIP, EEG, PPG, and/or GSR sensors altogether while crafting wearable multimodel sensing system are disclosed. FIG. 5 shows the overview of Painometry hardware schematic with the corresponding SIP sub-circuit 502, EEG sub-circuit 504, PPG sub-circuit 506, and/or GSR sub-circuit 508.

Safety consideration. Since the customized SIP and GSR sensors 102 and 106 are considered ‘active’ (i.e., they excite electrical signals to the part of the human body), special precautions need to be taken to ensure user's safety. According to the ICNIRP guidelines and IEEE C95.1 standard, 1 mA is considered as a safety threshold for DC and AC signals having a frequency below 1 MHz. As a result, in one embodiment, the SIP sensors 102 have been configured with a maximum output AC of 0.98 Vₚₚ and maximum output current of 0.25 mA. The GSR sensors 106 are designed to restrict only the DC signal of 1.2 V applied directly to human skin. Furthermore, the GSR sub-circuit 508 output current is limited at 0.5 mA by a current-limit resistor. Moreover, the AC signal from SIP is configured to not interfere with the brain rhythms. For example, in some embodiments, this results in a constraint that the AC signal for SIP is larger than 1 kHz.

Noise reduction. There are three main noise sources in the system: (1) electromagnetic noises from the surrounding environment coupling into the signal wires and human body, (2) parasitic noise from electrical components in sensing sub-circuits themselves, and (3) cross-talk noise induced by high-speed digital components into the analog domain. The EEG acquisition device (available from OpenBCI) provides a driven right leg circuit which helps to eliminate common-mode noise coupled into the human body. To further increase the signal-to-noise ratio, the amplifiers of the sensing circuits are placed close to the point of measurement, and the wires are kept as short as possible. Additionally, all of the electrical components used to construct Painometry hardware are high precision with low tolerance level.

Cross-talk noise is alleviated by ground separation in hardware design including (i) separation of the analog ground domain 602 and digital ground domain 604 and (ii) separation between each sub-circuit as depicted in FIG. 6. SIP measurements on two corrugator groups are processed sequentially to avoid the cross-talk interference from two AC sweep sources. In some embodiments, the SIP sub-circuit 502 utilizes an analog switch 510 to consecutively interchange the SIP measurement between those two muscle groups.

### 6. Objective Pain Quantification

6.1 Signal Pre-Processing

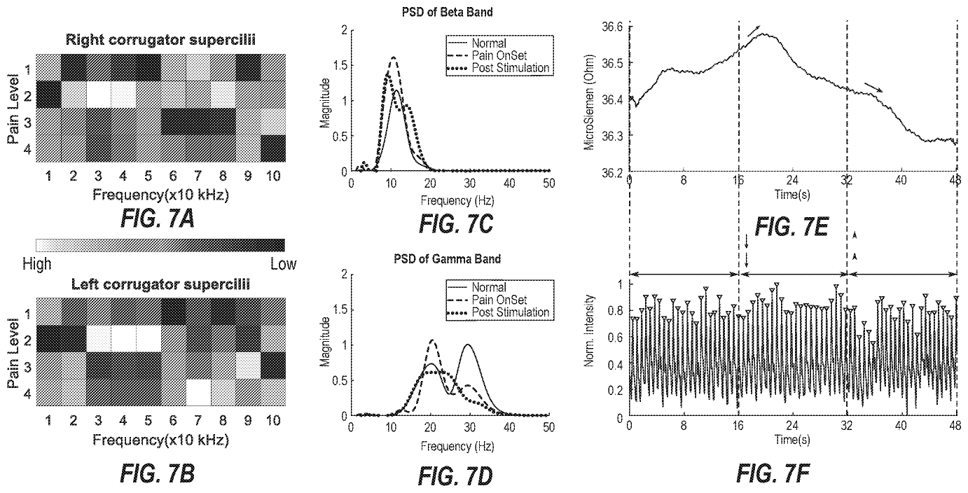

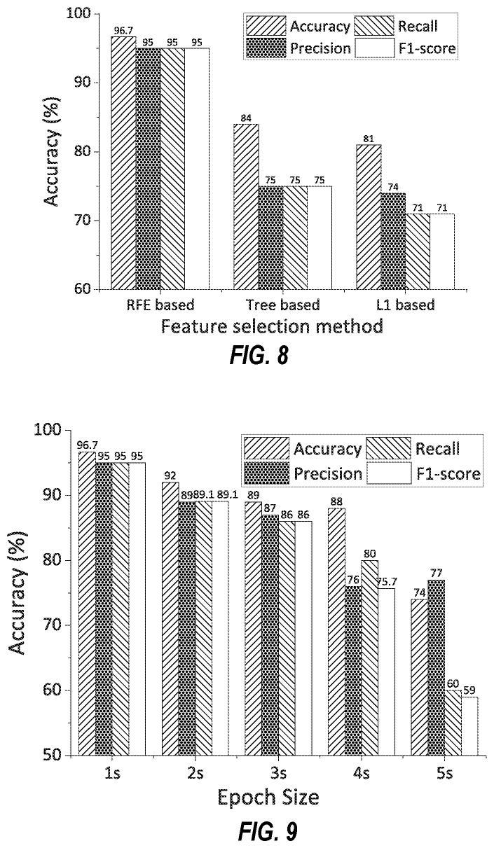

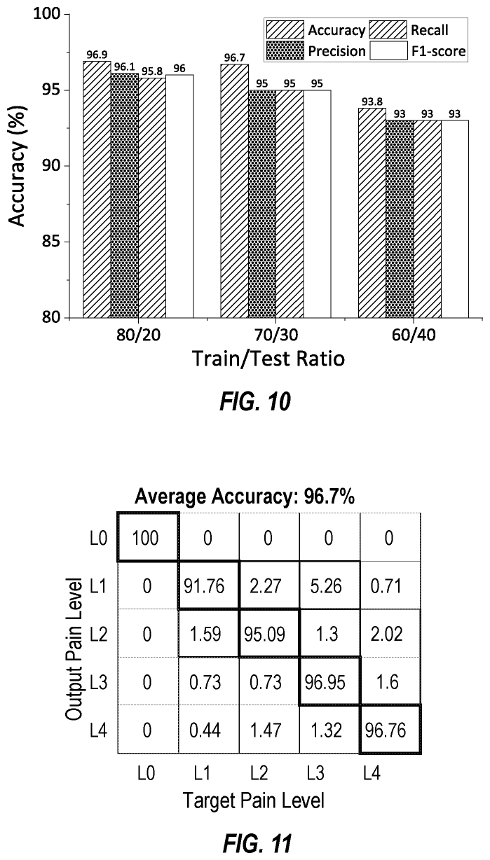

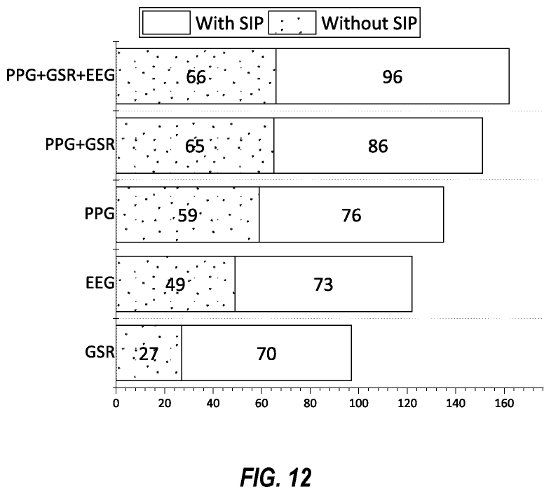

In Painometry, the signals collected from four sensing sources: SIP sensors 102, EEG sensors 104, GSR sensors 106, and PPG sensors 108 are analyzed. Specifically, the signals are pre-processed according to their properties before being put into a feature analysis pipeline. FIGS. 7A-F shows behaviors of the signals sensed under the influence of pain-inducing stimulation (details of the stimulation protocol is in Sec. 8 below). In particular, FIG. 7A illustrates normalized average SIP measurements with different pain levels for the right corrugator supercilii, FIG. 7B illustrates normalized average SIP measurements with different pain levels for the left corrugator supercilii, FIG. 7C illustrates a PSD envelope of a beta band in EEG from T4 montage, FIG. 7D illustrates a PSD envelope of gamma band in EEG from T4 montage, FIG. 7E illustrates GSR, and FIG. 7F illustrates PPG measurements with peak detection of HR/HRV trend in experimental pain simulation.

Signal cleaning process. Even with effort in minimizing hardware noise as stated in the previous section, the raw sensing data is still prone to other noises including (1) DC drifting, (2) 50/60 Hz power line interference, and (3) users' movements. Hence, embodiments may be configured to continuously process the noisy data using an overlapped sliding window. For example, embodiments may initially apply a spline interpolation algorithm 306 so that all measurements have the same length. Embodiments may then remove the DC linear trend (using a DC removal module 308) of each window (except GSR measurement) by subtracting the 6th-order polynomial fit from original signals. The reason GSR is left out is because the DC level in GSR has useful information used in the skin conductance level calculation 316. Finally, a notch filter 310 is applied to remove 50/60 Hz power line interference, a lowpass filter of 5 Hz (for the illustrated embodiment) for PPG, and 100 Hz filter (for the illustrated embodiments) for EEG signal to remove unexpected movements.

Wavelet decomposition 318 for EEG. Pain features spread on different regions of the frequency domain, i.e., alpha, beta, gamma, delta, theta. A single band does not fully capture the brain's response to pain. Therefore, decomposing signals into multiple frequency bands facilitates more distinctive features to be extracted. A bandpass filter 312 is a naive approach to extract different frequency bands. However, applying a bandpass filter causes drift problem, spoils the beginning part of the signals and loses the crucial details of the acute pain case. Using a Fourier transform is another common method that breaks down the signal into a sum of sinusoidal components. However, only frequency information is taken into account, which causes the loss of temporal information. Wavelet decomposition 318, on the other hand, balances the trade-off between temporal and frequency resolution. Thus, some embodiments apply a 6^{th} order Daubechies 9 wavelet decomposition to extract all five brain rhythms from the cleaned EEG signal.

Peak detection and HR/HRV algorithm 314. Each heartbeat creates a peak that can be seen clearly in each PPG window. Therefore, embodiments may apply a peak detection algorithm 314. The sub-window slides, overlapping, until a local peak above the magnitude threshold is found. Then, the sub-window skips the sliding over this heartbeat interval and looks for the local peak of the next heartbeat interval. The heart rate (HR) and heart rate variability (HRV) are calculated as follows:

$$\{ \begin{matrix}HR = \frac{60 * Peak Count}{Final Peak - First Peak} \\ HRV = δ ( All Adj Peak Distances )\end{matrix} \qquad ( 7 )$$

6.2 Pain Features Extraction Algorithm 320

Algorithm 1: Training Algorithm

| | |
|---|---|
| 1 | Input: IS-Input Signal PLL-Pain Level |
| | ST Segment Time |
| 2 | Output: Mode/Trained Model SF-Selected Features |
| 3 | A: Feature Extraction |
| 4 | Segs ← SegmentSignal(IS, ST); |
| 5 | for i = 1 → sizeof(Segs) do |
| 6 | \| FS ← GenerateFeatures(Segs [symbol] ); |
| 7 | B: Feature Selection |
| 8 | while FS != [symbol] do |
| 9 | //Calculate Feature Importance: |
| 10 | Ranking ← Trainmodel(F S, PLL); |
| 11 | //Drop the least important feature: |
| 12 | FS ← FS−min(Ranking) |
| 13 | SF ← Selected Features |
| 14 | TrainingSet ← SplitTrainTest[SF, 70, 30]; |
| 15 | model ← Trainmodel(TrainingSet, PLL); |

In the illustrated automatic pain level identification system, embodiments analyze the data from four source signals: EEG, GSR, PPG and SIP. After obtaining signals from the appropriate sensors, the collected time series data from each source is segmented to fixed-size epochs, and selected features are extracted from each epoch to be used for classification of the pain level in that epoch. Classifiers are trained in a supervised manner using labeled epochs and subsequently used for real-time classification of the pain level. In the rest of this section, a feature extraction algorithm 320 is presented. Subsequently in the following sections, a feature selection algorithm 322 and classification steps in the pain level quantification pipeline is presented. Algorithm 1 shows a sample execution of this pipeline including execution by one or more processors and computer executable instructions implemented on the processors (assuming the RFE method for feature selection, in this case). The features selected for extraction from each signal are from a variety of categories as follows:

Temporal features: This category includes typical features for time series data analysis in the original domain (i.e., temporal domain), namely, mean, variance, skewness, and kurtosis. In pain classification, both GSR and SIP signals are often analyzed in time domain due to their considerable variation in amplitude and lack of distinctive frequency patterns. Hence, those four temporal features are extracted from single GSR channel and ten channels of SIP. Furthermore, since heart rate changes during the pain, embodiments choose heart rate variability as one independent temporal feature. Overall, in some embodiments, there are 45 temporal features extracted (i.e., 4 GSR, 40 SIP, and 1 HRV).

Spectral features: The spectral features are extracted to analyze the characteristics of EEG signal because brain waves are generally available in discrete frequency ranges at different stages. By transforming the time series EEG signal into the frequency domain in different frequency bands (i.e., Theta, Alpha, Sigma, Beta, and Gamma) and computing its power spectrum density, various spectral features can be extracted. The spectral features are extracted from the EEG signals at T3, T4, Fp1, and Fp2 channels, which include the ratio of powers, absolute powers, theta/gamma, theta/alpha, and sigma/gamma. Accordingly, 13 features are extracted from each of the four channels of EEG (in the illustrated example), which provides 52 spectral features in total.

Non-linear features: Bioelectrical signals show various complex behaviors with nonlinear properties. In particular, the chaotic parameters of EEG can be used for pain level classification. The discriminant ability of nonlinear analyses of EEG dynamics is demonstrated through the measures of complexity such as correlation dimension, Lyapunov exponent, entropy, fractal dimension, etc., with the last two features proven to be most informative. These two non-linear features are extracted for each of the EEG channels (a total of 8 features). Table 1 summarizes the features extracted from each type of signal under each category of features.

TABLE 1
List of pain features

| Type | Signal | Feature |
|---|---|---|
| Temporal | GSR, SIP | mean, variance |
| | | skewness, kurtosis |
| Temporal | PPG | heart rate variability |
| Spectral | EEG | absolute spectral powers |
| | | relative spectral powers |
| | | relative spectral ratio |
| Non-linear | EEG | fractal dimension, entropy |

6.3 Pain Feature Selection Algorithm 322

Even though each extracted feature can capture certain characteristics of the input signals, the performance of a classification algorithm, implemented by one or more processors and computer executable instructions implemented on the processors, can be degraded when all possible features are used altogether, mainly due to feature redundancy. In particular, some of the features may be irrelevant or redundant, which further reduces the classification accuracy.

In order to select a set of relevant features among the 105 extracted features, the discriminating ability of each feature is computed when they are used in combination. However, it is computationally infeasible to test all of the possible combinations. As a result, some embodiments adopt three feature selection methods, namely, Recursive Feature Elimination (RFE), L1-based, and treebased feature selection, as alternatives to identify the most effective combination of features. A greedy optimization algorithm, implemented by one or more processors and computer executable instructions implemented on the processors that seeks to improve generalization performance of the classification model by removing the least important features whose deletion will have the least effect on training error can be used. L1 based feature selection is used for linear models including Logistic Regression and SVM. Since these linear models are applied during the classification process, L1 norm is used to remove features with zero coefficients. Finally, with tree-based feature selection one can take a different approach by computing importance of features which in turn is used to remove irrelevant features.

6.4 Pain Level Quantification Algorithm 324

Various classification methods including the Support Vector Machine (SVM), Logistic Regression, Decision Tree and Random Forest can be used for pain or stress level detection, each shown to be effective in specific settings.

### 7. Implementation

From the discussion in Sec. 4, Painometry hardware (FIG. 6) is implemented with the design considerations for safety and noise mitigation.

SIP sensor hardware. An SIP sensor is built from the high-resolution impedance analyzer AD5933 and the low noise op-amp AD8606, both available from Analog Devices, Inc. of Norwood, Mass. To measure SIP from two corrugator muscles sequentially, embodiments may use, e.g., the analog switch TS5A23159, available from Texas instruments Inc., of Dallas, Tex., and four electrode montages over eyebrows (e.g., as shown in FIG. 1). The SIP sensors and sub-circuits, in the illustrated example, sweeps 10 excitation signals over the range of 10 kHz to 100 kHz at 0.98 Vpp and maximum output current of 0.25 mA.

GSR sensor hardware. In the illustrated example, GSR sensors and sub-circuits are built based on a non-inverting amplifying circuit with the single micro-power precision amplifier LMP2231 and a precision micro-power shunt voltage reference LM4041, both available from Texas instruments, Inc. of Dallas, Tex. The GSR sensors use two electrode montages on top of the forehead, such as is illustrated in FIG. 1. The gain resistor may be tuned so that the swing range of GSR output expands as much as possible in the ADC reading range of the main MCU 512. The GSR sub-circuit 508 has output current limited at 0.5 mA by a current-limit resistor.

The GSR sensor uses two electrode montages on top of the forehead as illustrated in FIG. 1. The gain resistor may be tuned so that the swing range of GSR output expands as much as possible in the ADC reading range of the main MCU 512. The GSR sub-circuit 508 output current is limited at 0.5 mA by a current-limit resistor.

PPG sensor hardware. A pulse sensor is used, such as one available from World Famous Electronics, LLC. In this design, there is a super-bright green LED used with the dominant wavelength at 525 nm accompanied by a highly sensitive light sensor to pick up reflected light from the artery. The green LED and light sensor are placed in the middle of the forehead as discussed in section 2, and as illustrated in FIG. 1.

EEG sensor hardware. Some embodiments use the OpenBCI EEG acquisition device to capture electrical brain activities. This device can check the electrodes-skin contact impedance using a lead-off detection method to ensure high-quality signals before the measurement. Embodiments may also use this feature to measure the ZE1S and ZE2S in the SIP circuit model to calibrate SIP measurements beforehand. The EEG hardware uses ten electrode montages including 8 EEG channels (T3-T4, Fp1-Fp2, F7F8, and O1-O2) and reference and bias placed at T5 and T6 channels, respectively.

Putting together Painomerty. Some embodiments use the ultralow power TI MSP430F5529 available from Texas Instruments, Inc. of Dallas, Tex., as the main MCU 512 which connects to SIP sensor 102 via I2C and to the GSR and PPG sensors 106 and 108 via analog I/O. Sensor measurements are then broadcasted out wirelessly via a CC2650 BLE module, which is a Bluetooth module available from Texas Instruments, Inc. of Dallas, Tex. Some embodiments use gold-cup electrodes for all sensor montages. In some embodiments, Ten20 conductive paste, available from Weaver and Company, of Aurora, Calif., is applied to the sensors while collecting data to ensure low impedance at electrode-skin contact. Electrodes and hardware PCBs are mounted onto a hook and loop fastener flexible headband.

### 8. Performance Evaluation and Comparative Examples

A set of experiments was conducted to evaluate the overall performance of Painometry. Also, the SIP sensor performance was evaluated in order to demonstrate its ability to capture the autonomous muscle movements. In particular, the following aspects were evaluated: (1) reliability and safety of the experiment protocol, (2) performance of pain quantification pipeline, and (3) user experience survey.

8.1 Experimental Methodology

Experiments were conducted for (1) inducing clearly distinguishable pain levels in order to get true labels for the groundtruth of 5 different pain states (i.e. no pain and 4 pain levels), (2) avoiding the hypoalgesia phenomenon in which the subjects get familiar to the pain and experiencer reduced pain sensation, and (3) ensuring the safety of the experimental conditions. Participant demographics are shown in Tab. 2. All subjects in the study were pre-screened to be pain-free at the time of experiment, i.e. no chronic pain or recent injury.

TABLE 2
Data collection details.
Participant Demographics

| | |
|---|---|
| Age (years) | 21 52 years old |
| Gender Ratio | Male: 24, Female: 7 |
| Pain states | 5 (pain-free and 4 levels of pain) |
| Data collection | 16 pain-inducing runs, random order |
| Bruise after experiment | 9.7% (3 subjects) |

Existing pain-related research has used various methods to induce experimental pain such as pressure pain, thermal pain, and cold pain. Each method has its own unpleasantness and side effects after the experiment (e.g. bruise after pressure pain, skin burnt after thermal pain or allergy after cold pain). Pressure pain induced on a subjects' thumb was selected as the experimental-pain method because the development of acute pain with clear distinction between different pain levels can be guaranteed. Further, this method has low probability of producing side effects (e.g., no bruises reported from 12 subjects in a pilot study and only 3 reported from 31 subjects in the research study).

The pressure pain device (PPD) is designed to be safe and accurate in delivering experimental pain. This device includes the mechanical pain delivery part (with a piston-like front-end), the pressure controller hardware and a compressed air tank. The device is designed with a handle to keep the hand comfortable and still while receiving pressure. A hardware pressure regulator limits pressure to below 14 kg/cm2 to prevent excessive pressure. Pressure intensity and duration are controlled by a LabView implementation. PPD tracks both valve pressure level and piston-to-thumbnail pressure level in real-time. To ensure the safety of the device and experimental protocol, the mechanical pain delivery part is held together by two asymmetrical screws which gives subjects the capability to remove their thumbs at their discretion.

The pain stimulation protocol is designed to deliver clear distinct pain levels to the subject and to avoid the hypoalgesia effect, whereby a decreased sensitivity to painful stimuli occurs. PPD was used to create 4 different pain levels of mild, moderate, strong, and severe pain (denoted as L1-L4 in the remaining parts of this section), which correspond to the levels of 1-2, 2-4, 4-6, and 6-7 in the VAS scale. Each subject will experience 16 stimulation runs (i.e. 4 runs for each pain level) in a pseudo-random sequence. Each run is divided into 3 intervals: T1 (before the stimulus) is first and lasts 16 seconds, T2 (pain stimulus onset) is next and lasts 16 seconds, and T3 (after the pain stimulus ceased) lasts in 30 seconds at least. During the experiment, the Painometry headband records the subjects' biosignals through all the runs. The subjects rate their pain subjectively using the VAS scale right after T2. In addition, T3 is adjusted on-the-fly in order to ensure the subjects' thumb returns to normal feeling before each stimulation run. The pressure applied on the subjects' thumbnail scales proportionally to 4 valve pressure levels. The average pain rating shows the effect of pressure levels to the subjects' rating on stimulation runs.

8.2 System Performance

The performance of the pain quantification pipeline was evaluated by analyzing the correlation of features with pain levels and explaining the result of different feature selection methods on the data with 1 second epoch size. After selecting the most distinguishing features, the performance of four classifiers are determined by computing the accuracy, recall, and precision under different conditions: epoch size in seconds, training data size, and sensor(s) used.

TABLE 3
Comparison of pain classification
performance of different methods.

| Method | Precision | Recall | F1 |
|---|---|---|---|
| LR | 0.42 | 0.41 | 0.37 |
| DT | 0.79 | 0.72 | 0.73 |
| SVM | 0.66 | 0.64 | 0.64 |
| RFC | 0.95 | 0.95 | 0.95 |

To study the expressive power of the features extracted in the pipeline, the correlation between each of the extracted features and the pain levels was calculated. The features extracted from the SIP signal have the highest correlation with the pain levels with a mean of 0.6. Features from the EEG, PPG, and GSR signals ranked in the second, third, and fourth, respectively.

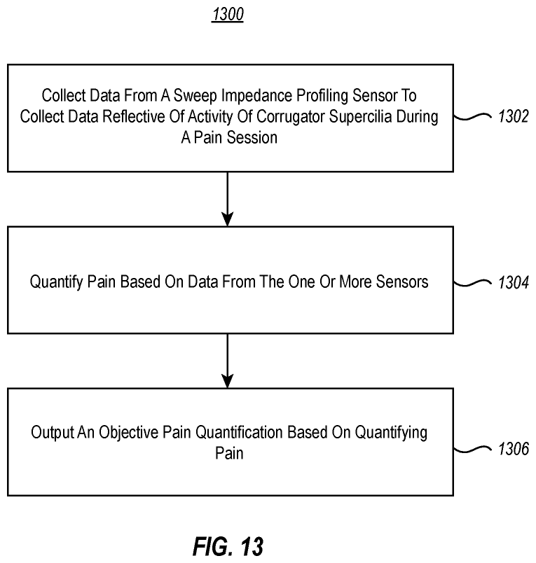

The performance of various feature selection methods were evaluated. FIG. 13 displays the accuracy when the features are selected from the RFE, Treebased, and L1-based approaches. The result shows that RFE with Random Forest provides the best accuracy of 96.7% among the combination of feature selection methods and classifiers. The top 15 features are listed in the Table 4, which give the highest accuracy. Features with the most distinguishing power include the magnitude of the SIP signal, heart rate variability, EEG signals at T3 and T4 channels, and GSR. These were selected as the final feature sets for the rest of the evaluation.

TABLE 4
List of selected features

| Type | Signal | Feature |
|---|---|---|
| Temporal | GSR, SIP | GSR (mean), SIP (1, 2, 4, 5, 6, 8, 9) |
| Temporal | PPG | heart rate variability |
| Spectral | EEG | Absolute (Beta) (T3) |
| | | Absolute (Gamma) (T3) |
| | | Relative (Gamma) (T3) |
| Non-linear | EEG | Entropy |

After selecting the features with the most discriminating power, the results of the pain level classification were evaluated with 10-fold cross-validation for training and evaluation. For each split, 30% of data samples are held out as test data. Table 3 illustrates the results for different classification methods including Logistic Regression (LR), Decision Tree (DT), Support Vector Machines (SVM), and Random Forest Classifier (RFC).

As illustrated, the maximum accuracy of 96.7% is achieved using RFC among different classification methods. On the other hand, the LR method provides the least accuracy of 63%. To improve the accuracy of the RFC model, the hyperparameters were further tuned and an optimal setting selected to achieve the maximum accuracy. The optimal hyperparameters were found to be 30 estimators (trees), a maximum tree depth of 15, and square root of the total number of features for the maximum number of features selected for each tree. After selecting the optimal setting, the model was evaluated for different epoch sizes (1, 2, 3, 4, and 5 seconds) and train and test split ratios as shown in FIGS. 14 and 15, respectively. As illustrated, the result of classification is significant when considering 1-second and 2-second epoch sizes since acute pain is being evaluated.

Finally, FIG. 11 presents the confusion matrix obtained with the tuned RFC model. The columns represent the pain levels predicted by the classifier and the rows represent the true pain levels. Although some pairs of the pain levels such as L1 and L2 as well as L3 and L4 are very close and might be prone to misclassification, the final quantification model after feature selection and hyperparameter tuning can efficiently distinguish all the pain levels.

In the last experiment represented by FIG. 17, how using different combinations of biosignal sensors would impact the classification accuracy was studied. As illustrated, using only GSR and PPG signals can classify the pain levels with accuracy of 27% and 59%, respectively. On the other hand, considering only EEG and three signals of PPG, GSR and EEG without SIP signal can achieve an accuracy of 49% and 66%, respectively. Finally, by combining all the four signals, a maximum accuracy of 96.7% was achieved. This illustrates a surprising an unexpected result by being able to accurately measure pain with a much higher accuracy when SIP is used than when only conventional pain quantification methods are used.

8.3 User Study

While developing and evaluating Painometry, a survey was conducted to identify gaps in the technologies and people's expectation. The survey was distributed to 35 people, including the 31 subjects and 4 people who hadn't experienced Painometry yet. FIG. 16 shows five questions that were asked and the statistics on the participants' answers. Specifically, analyzing the statistic result of Question 1 shows that objectively rating the pain is not easy for everyone. From Question 2-3, it can be seen that 11 out of 35 participants experienced pain within a week; and nearly 80% of them struggled to rate it. Generally speaking, an objective pain quantifier is a need that will be very helpful to avoid the inappropriate dosage of pain relievers used, which usually harms people's health in the long term and can lead to dependency after use. Finally, the duration which people are willing to tolerate a headband-like device with the placement of sensors on their forehead and around their head during the use was studied. Statistically, Question 4-5 recommends that people feel comfortable wearing such a device and sensors 15-30 minutes at most. Consequently, this information can be used to build a timing constraints in the a pain intervention system.

### 9 Specific Example Embodiments

Referring to FIG. 13, a method is illustrated. The method includes acts for objectively measuring pain. The method 1300 includes collecting data from one or more sensors in a headband, including collecting data from a sweep impedance profiling sensor to collect data reflective of activity of corrugator supercilia during a pain session (act 1302).

The method 1300 further includes objectively quantifying pain based on data from the one or more sensors (act 1304).

The method 1300 further includes outputting an objective pain quantification based on quantifying pain (act 1306).

The method 1300 may further include placing the sweep impedance profiling sensor over corrugator supercilia muscles.

The method 1300 may further include causing the sweep impedance profiling sensor to have an output AC of 0.98 Vₚₚ and maximum output current of 0.25 mA.

The method 1300 may further include causing the sweep impedance profiling sensor to have an AC output at or above 1 kHz.

The method 1300 may further include processing measurements on two corrugator groups sequentially.

The method 1300 may further include sending data from the one or more sensors wirelessly.

The method 1300 may further include using the objective pain quantification to assist in prescribing correct pain medication dosing for patients who suffer from chronic pain.

The method 1300 may be practiced where collecting data from one or more sensors in a headband, comprises collecting data from at least one of: an electromyography sensor to collect data signals from a brain that reflects pain perception; a photoplethysmogram sensor configured to collect data regarding changes in hear rate and heart rate variability due to pain state; or a galvanic skin response sensor configured to collect data related to changes in sweat gland and skin conductance due to a pain state.

One embodiment of the invention includes a wearable objective pain measuring device. The wearable objective pain measuring device includes a headband configured to be worn by a user on the user's head. The wearable objective pain measuring device includes one or more sensors in the headband. The one or more sensors include a sweep impedance profiling sensor to collect data reflective of activity of corrugator supercilia during a pain session. The wearable objective pain measuring device includes a pain quantification pipeline configured to objectively quantify pain based on data from the one or more sensors. The wearable objective pain measuring device includes an output device configured to output an objective pain quantification based on quantifying pain.

In one aspect the wearable objective pain measuring device is implemented wherein the sweep impedance profiling sensor configured to be placed over corrugator supercilia muscles.

In one aspect the wearable objective pain measuring device is implemented wherein the sweep impedance profiling sensor is configured to have an output AC of 0.98 Vₚₚ and maximum output current of 0.25 mA.

In one aspect the wearable objective pain measuring device is implemented wherein the sweep impedance profiling sensor is configured to have an AC output at or above 1 kHz.

In one aspect the wearable objective pain measuring device is implemented wherein the sweep impedance profiling sensor is configured to process measurements on two corrugator groups sequentially.

In one aspect the wearable objective pain measuring device is implemented wherein the device comprises separate digital and analog ground planes.

In one aspect the wearable objective pain measuring device is implemented wherein the device is wireless.

In one aspect the wearable objective pain measuring device is implemented wherein the device is configured to be used to assist in prescribing correct pain medication dosing for patients who suffer from chronic pain.

In one aspect the wearable objective pain measuring device further comprises at least one of: an electromyography sensor to collect data signals from a brain that reflects pain perception; a photoplethysmogram sensor configured to collect data regarding changes in hear rate and heart rate variability due to pain state; or a galvanic skin response sensor configured to collect data related to changes in sweat gland and skin conductance due to a pain state.

One embodiment of the invention includes a computing system comprising one or more processors. The computing system further comprises one or more computer-readable media having thereon computer-executable instructions that are structured such that, when executed by the one or more processors, cause the computing system to objectively measure pain, including instructions that when executed by the one or more processors cause the computing system to perform the following: collect data from one or more sensors in a headband, including collecting data from a sweep impedance profiling sensor to collect data reflective of activity of corrugator supercilia during a pain session; objectively quantify pain based on data from the one or more sensors; and output an objective pain quantification based on quantifying pain.

In one aspect, the computing system is implemented where the one or more computer readable media further comprising computer-executable instructions that are structured such that, when executed by the one or more processors, cause the computing system to process measurements on two corrugator groups sequentially.

In one aspect, the computing system is implemented where collecting data from one or more sensors in a headband, comprises collecting data from at least one of: an electromyography sensor to collect data signals from a brain that reflects pain perception; a photoplethysmogram sensor configured to collect data regarding changes in hear rate and heart rate variability due to pain state; or a galvanic skin response sensor configured to collect data related to changes in sweat gland and skin conductance due to a pain state.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

Embodiments of the present invention may comprise or utilize a special purpose or general-purpose computer including computer hardware, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A wearable objective pain measuring device comprising:
a headband configured to be worn by a user on the user's head;
one or more sensors in the headband, the one or more sensors comprising:
a sweep impedance profiling sensor to collect data reflective of activity of corrugator supercilia during a pain session;
a pain quantification pipeline configured to objectively quantify pain based on data from the one or more sensors; and
an output device configured to output an objective pain quantification based on quantifying pain.

2. The wearable objective pain measuring device of claim 1, wherein the sweep impedance profiling sensor configured to be placed over corrugator supercilia muscles.

3. The wearable objective pain measuring device of claim 1, wherein the sweep impedance profiling sensor is configured to have an output AC of 0.98 Vₚₚ and maximum output current of 0.25 mA.

4. The wearable objective pain measuring device of claim 1, wherein the sweep impedance profiling sensor is configured to have an AC output at or above 1 kHz.

5. The wearable objective pain measuring device of claim 1, wherein the sweep impedance profiling sensor is configured to process measurements on two corrugator groups sequentially.

6. The wearable objective pain measuring device of claim 1, wherein the device comprises separate digital and analog ground planes.

7. The wearable objective pain measuring device of claim 1, wherein the device is wireless.

8. The wearable objective pain measuring device of claim 1, wherein the device is configured to be used to assist in prescribing correct pain medication dosing for patients who suffer from chronic pain.

9. The wearable objective pain measuring device of claim 1, further comprising at least one of:
an electromyography sensor to collect data signals from a brain that reflects pain perception;
a photoplethysmogram sensor configured to collect data regarding changes in hear rate and heart rate variability due to pain state; or
a galvanic skin response sensor configured to collect data related to changes in sweat gland and skin conductance due to a pain state.

10. A method of objectively measuring pain, the method comprising:
collecting data from one or more sensors in a headband, including collecting data from a sweep impedance profiling sensor to collect data reflective of activity of corrugator supercilia during a pain session;
objectively quantifying pain based on data from the one or more sensors; and
outputting an objective pain quantification based on quantifying pain.

11. The method of claim 10, further comprising placing the sweep impedance profiling sensor over corrugator supercilia muscles.

12. The method of claim 10, further comprising causing the sweep impedance profiling sensor to have an output AC of 0.98 Vₚₚ and maximum output current of 0.25 mA.

13. The method of claim 10, further comprising causing the sweep impedance profiling sensor to have an AC output at or above 1 kHz.

14. The method of claim 10, further comprising processing measurements on two corrugator groups sequentially.

15. The method of claim 10, further comprising sending data from the one or more sensors wirelessly.

16. The method of claim 10, further comprising using the objective pain quantification to assist in prescribing correct pain medication dosing for patients who suffer from chronic pain.

17. The method of claim 10, wherein collecting data from one or more sensors in a headband, comprises collecting data from at least one of:
an electromyography sensor to collect data signals from a brain that reflects pain perception;
a photoplethysmogram sensor configured to collect data regarding changes in hear rate and heart rate variability due to pain state; or
a galvanic skin response sensor configured to collect data related to changes in sweat gland and skin conductance due to a pain state.

18. A computing system comprising:
one or more processors; and
one or more computer-readable media having thereon computer-executable instructions that are structured such that, when executed by the one or more processors, cause the computing system to objectively measure pain, including instructions that when executed by the one or more processors cause the computing system to perform the following:
collect data from one or more sensors in a headband, including collecting data from a sweep impedance profiling sensor to collect data reflective of activity of corrugator supercilia during a pain session;
objectively quantify pain based on data from the one or more sensors; and
output an objective pain quantification based on quantifying pain.

19. The computing system of claim 18, wherein the one or more computer readable media further comprising computer-executable instructions that are structured such that, when executed by the one or more processors, cause the computing system to process measurements on two corrugator groups sequentially.

20. The computing system of claim 18, wherein collecting data from one or more sensors in a headband, comprises collecting data from at least one of:
an electromyography sensor to collect data signals from a brain that reflects pain perception;
a photoplethysmogram sensor configured to collect data regarding changes in hear rate and heart rate variability due to pain state; or
a galvanic skin response sensor configured to collect data related to changes in sweat gland and skin conductance due to a pain state.
